(19) United States
(12) Patent Application Publication
(10) Pub. No.: US 2003/0211157 A1
(43) Pub. Date: Nov. 13, 2003

(54) Semi-sol delivery blend for water soluble molecules

(76) Inventor: David Lew Simon, Mansfield Center, CT (US)

Correspondence Address: David L. Simon, 100 Cemetery Road, P.O. Box 618, Mansfield Center, CT, 06250, US

(21) Appl. No.: 10/306,657

(22) Filed: Nov. 27, 2002

Related U.S. Application Data
(63) Continuation-in-part of application No. 09/922,873, filed on Aug. 6, 2001, now Pat. No. 6,569,866.
Application No. 09/922,873 is a continuation-in-part of application No. 09/152,834, filed on Sep. 14, 1998, now Pat. No. 6,271,240.
Application No. 09/152,834 is a continuation-in-part of application No. 08/866,334, filed on May 30, 1997, now abandoned.
Application No. 09/152,834 is a continuation-in-part of application No. 08/643,775, filed on May 6, 1996, now abandoned.

(51) Int. Cl. A61K 31/573; A61K 31/485; A61K 9/14

(52) U.S. Cl. 424/486; 514/282; 514/179

(57) ABSTRACT

The present invention relates to novel methods and compositions for administration of water-soluble small molecules for the treatment of opioid addiction, alcoholism and HIV infection, especially where the water-soluble small molecules are pentacyclic and structurally similar to nalmefene, buprenorphine, 6-beta-naltrexol and nalbuphine.

### RELATED APPLICATIONS

This application is a continuation-in-part of co-pending U.S. patent application Ser. No. 09/922,873 filed Aug. 6, 2001, which is a continuation-in-part of U.S. patent application Ser. No. 09/152,834 filed Sep. 14, 1998, which is a continuation-in-part of U.S. patent application Ser. No. 08/866,334 filed May 30, 1997 and U.S. patent application Ser. No. 08/643,775 filed May 6, 1996, the disclosure of each which is hereby incorporated by reference.

### FIELD OF INVENTION

The present invention relates to novel pharmaceutical preparations for sustained delivery of water-soluble drugs or molecules to a human or animal. This invention may be used to deliver nalmefene hydrochloride to humans for the treatment of compulsive behaviors as may be associated with alcohol, cocaine or opioid dependencies, and other water-soluble molecules structurally similar to nalmefene including buprenorphine, 6-beta-naltrexol and nalbuphine. These compositions may be used to treat HIV infection as well.

### BACKGROUND OF THE INVENTION

An opioid agonist analgesic is a drug or pharmaceutical agent that traditionally is used to treat pain, to suppress coughing, to treat diarrhea, and for other medicinal uses. Depending upon the degree with which a particular opioid agonist medication binds to specific opioid receptor subtypes, such as its affinity for one opioid subtype receptor in preference to another, the opioid agonist analgesic may tend to cause euphoria, or it may tend to cause dysphoria. Some opioid analgesic agonists may also tend to cause nausea by stimulating or inhibiting areas in the brain known as “the vomiting center” and “the chemotactic zone,” depending upon the degree with which specific opioid receptor subtypes are activated, and depending to some extent upon the ability of a particular opioid agonist analgesic to penetrate the blood-brain-barrier (BBB). Examples of opioid receptor subtypes are delta-receptors, kappa-receptors, mu-receptors and sigma receptors. These opioid receptor subtypes may be further subcategorized, as for example, mu₁-receptors and mu₂-receptors.

The opioid antagonist nalmefene has unique characteristics which set it apart from other opioid antagonists such as, for example, naloxone and naltrexone. The unique opioid receptor subtype binding profile of nalmefene enables nalmefene alone, as compared to naloxone and naltrexone, to allow preferred antagonism of opioids at the kappa-opioid receptors versus the mu-opioid receptors, which in turn results in an optimal homeostatic balance of dopamine.

Szekely shows a schematic representation of two opposing opioid systems located in the mesolimbic system of the human central nervous system. These systems modulate A10 dopaminergic neurons projecting in the nucleus accumbens. As illustrated in this reference, stimulation of mu-opioid receptors (the mu subtype of opioid receptor) in the ventral tegmental area (VTA), the site of origin of the A10 neurons, increases dopamine release in the nucleus accumbens (NA). Selective blockade of this mu-receptor results in significant decrease in dopamine release in the nucleus accumbens. In stark contrast, stimulation of kappa-receptors (the kappa subtype of opioid receptor) in either the VTA or the NA results in a decrease in the amount of dopamine released. Selective blockade of kappa-receptors significantly increases dopamine release.

Spanagel et al. demonstrate that tonically active and functionally opposing mu and kappa opioid systems regulate mesolimbic dopamine release in the nucleus accumbens. They report that the injection of mu-opioid agonists such as DAGO into the VTA stimulate mu-opioid receptors and increase the release of dopamine from the VTA into the NA. As would be expected, administration of a mu-opioid receptor antagonist into the VTA decreases dopamine release. The authors further report that kappa-opioid receptors agonists such as U-6953 infused into the NA inhibit dopamine release there, whereas kappa-opioid receptor antagonists such as nor-BNI increase dopamine release. An “agonist” is a “like” chemical with similar action to a given drug. An “antagonist” is a chemical, often with a similar chemical structure to a given drug, which exerts a dissimilar action to the given drug, in general preventing the “like” action of that given drug. With opioid receptors, in general, an agonist binds to the receptor and activates it in such a way as to begin a cascade of chemical or pharmacological events so as to result in the end effect related to a particular opioid receptor subtype. In contradistinction, an antagonist will bind to the receptor but not activate it. An antagonist exerts its actions by blocking the receptors from agonists, by physically occupying the space on the receptor where an agonist would otherwise bind.

The opposing mu and kappa opioid systems acting together provide a homeostasis of dopamine levels within the central nervous system. Changes in these opioid systems, such as by activation or blockade of the specific receptors, would therefore be expected to modulate opioid-induced effects that are mediated by mesolimbic pathways. Mu and kappa receptors are found elsewhere in the human body. For example, they have been located in the spinal cord (See Fujimoto, Bakshi and Behrmann, below) and in other non-central nervous system organs such as the kidney and intestine (See Ohnishi and Kreek, below). Accordingly, the model presented provides a neurochemical framework for understanding the adaptive changes resulting from long term use of opioids, as well as the clinical response elicited by exogenously administered opioid agonists and antagonists having different binding profiles.

For example, Pan et al report modifications in opioid-induced behavior resulting from changes in these mu and kappa systems. These authors state that the effects of opposing mu and kappa receptors extend to opioid action on emotion, perception and drug reinforcement. While morphine and other mu-opioid agonists increase dopamine release and produce euphoria and place preference, kappa-opioid agonists reduce mesolimbic dopamine release and produce dysphoria and aversion.

Scientists have shown that nalmefene, relative to other opioid antagonists such as naloxone and naltrexone, is significantly more kappa-receptor preferring. By way of example, Kreek et al. conclude that nalmefene has more kappa binding activity than either naloxone or naltrexone. Specifically, nalmefene is more potent than either naloxone or naltrexone as a kappa-receptor antagonist, and therefore would block kappa agonists (e.g. the naturally occurring dynorphin) to a greater extent than the other antagonists.

Fujimoto et al. demonstrate differences between mu and kappa receptor effects in the spinal cord. Specifically, these authors report that the administration of dynorphin, a potent kappa agonist, results in decreased analgesia. The dynorphin causes antianalgesic effects at the level of the spinal cord. Fujimoto shows that when a kappa-opioid receptor antagonist such as Cholera Toxin is given, the antianalgesic effect of dynorphin is inhibited.

Bakshi et al. shows that kappa receptors are widely distributed in the spinal cord, and that administration of dynorphin causes motor impairment. These authors also demonstrate that nalmefene is selective for these intraspinal kappa receptors, and limits dynorphin induced motor dysfunction after spinal cord injury.

Behrmann et al. report that a single dose of nalmefene has increased activity at kappa receptors and that a single dose of nalmefene exerts a significant neuroprotective effect after acute spinal cord injury, in direct contrast to the mu-preferring opioid antagonist naloxone that showed no significant effect on neurological recovery after spinal cord injury.

Ohnishi et al. teach the effects on urine production due to kappa-opioid receptor pharmacology at both the level of the pituitary gland and the kidney.

Crain et al. (U.S. Pat. No. 5,580,876) teach a method for “selectively enhancing the analgesic potency of a bimodally-acting opioid agonist” which shows that nalmefene, much more so than other opioid antagonists, enhances analgesia produced by opioid agonist analgesics. Crain et al. further teach that much lower concentrations of nalmefene are required to enhance analgesia than with either naloxone or naltrexone, thus further supporting that nalmefene optimizes dopamine homeostasis to a much greater extent than other opioid antagonists such as naloxone and naltrexone.

The prior art contains many examples of methods for prolonged delivery of naltrexone. Naltrexone implants, depots and other sustained release formulations of naltrexone have be described in great detail. These naltrexone preparations have been proposed as improved methodologies for treating addiction to opioid agonist analgesics. What has not been appreciated in the prior art are the unique pharacological and clinical advantages provided by the prolonged administration of nalmefene via sustained delivery formulations such as sustained release formulations for per os administration, subcutaneous implants, injected depot preparations for subcutaneous or intramuscular administration and transdermal delivery systems.

A significant problem in treating humans addicted to opioid agonist analgesics with per os naltrexone is the significant gastrointestinal upset which is often caused soon after per os administration of this drug. Thus, to encourage use of opioid antagonists for addiction treatment, it is important to formulate a delivery system of opioid antagonist that is administered in other than per os form. Such a delivery system would tend not to dissuade a human from being administered an opioid antagonist, even if it were not in a sustained delivery formulation. Examples of such delivery routes are buccal, intranasal, sublingual, transdermal and transmucosal preparations, including suppositories for rectal administration. These routes of delivery, even if not delivered over a very prolonged time, still would increase patient compliance with opioid antagonist administration by allowing a third party to administer, or to observe self-administration, of the opioid antagonist. For example, a “squirt” through the nares and onto the nasal mucosa would ensure a delivered dose of antagonist. Further, by bypassing the gastrointestinal tract, such intranasal administration is much less likely to cause gastrointestinal upset. Intranasal administration has the further advantage, as does sublingual administration, of bypassing metabolism by the liver upon initial administration. Metabolism of a drug by the liver after delivery to the gastrointestinal tract is generally referred to as “first pass metabolism,” and is a significant disadvantage for per os administration of many drugs. Nalmefene and naltrexone are two drugs that undergo very significant first pass metabolism. Of these two drugs, nalmefene is very much preferred for the treatment of opioid addiction because of its unique opioid receptor subtype binding profile compared to naltrexone, as described above.

The administration of opioid antagonists cause upregulation of opioid receptors present on the surface of cell of the central nervous system. The result of this increased density of opioid receptors is that more opioid receptors will then be available to the naturally occurring endogenous endorphins that are in proximity to these receptors. Because betaendbrphin production is decreased by a mechanism generally known as “negative feedback inhibition” in humans who are chemically dependent upon, and who are still being administered, exogenous opioid agonist analgesics, immediately upon cessation of opioid agonist analgesic administration there is a lack of beta-endorphin in these humans relative to the normal state in humans not chemically dependent upon opioid agonist analgesics. Thus, administration of opioid antagonists not only increase the number of receptors for beta-endorphin to bind to, in addition, these antagonists actually stimulate the production of endorphins by causing the release of negative feedback inhibition of its production. Thus, the cellular changes induced from chronic use of opioid agonist analgesics are reversed to a significant extent. Beta-endorphin attaches to and activates mu-opioid receptors, which results in a cascade of biochemical reactions, the result of which is an increase in central nervous system (CNS) dopamine. These changes brought upon by treatment with an opioid antagonist, such as nalmefene, restore to a human being a more normal physiological state, which will decrease the human's cravings for, and reduce the human's tolerance to, exogenously administered opioid agonist analgesics.

This upregulating effect of opioid antagonists in humans for treating addiction to opioid agonist analgesics has not been appreciated by those skilled in the art, particularly in the case of nalmefene which provides distinct pharacological and clinical advantages over other opioid antagonist for treating addiction to opioid agonist analgesics. Nalmefene tends to optimize CNS dopamine by virtue of its greater affinity for kappa-opioid receptors relative to mu-opioid receptors, as compared to naltrexone and other opioid antagonists.

A sufficiently high concentration of opioid antagonist must be present at the opioid receptor blocked, e.g. at a mu₁-opioid receptor, to prevent an exogenously administered opioid agonist analgesic or its metabolite from binding to the receptoror, but not such a high concentration as to totally block binding of endogenous beta-endorphin to that receptor. Again, nalmefene is the unique opioid antagonist which will block beta-endorphin at mu₁-opioid receptors to a relatively lesser extent than other antagonists such as naloxone and naltrexone, while at the same time having optimal blocking of kappa-opioid receptors by endogenous molecules such a dynorphins. Therefore, nalmefene alone, as compared to naloxone and naltrexone, not only optimizes dopamine regulation during detoxification, but also following detoxification. Thus, nalmefene is not an analogous compound to other opioid antagonists because nalmefene provides distinct pharmocological and clinical advantages for post detoxification treament of patients addicted to opioid narcotics not available with other opioid antagonists.

### SUMMARY OF THE INVENTION

The present invention comprises methods of administering the medicinal agent nalmefene, with or without co-administration of a centrally-acting dopaminergic drug such as bupropion. In one aspect, the invention provides a method for administering nalmefene which acts to produce a prescribed serum concentration of nalmefene over some time period that optimally regulates dopamine release in the central nervous system. In a second aspect, the invention provides a method for administering nalmefene which bypasses the gastrointestinal tract and therefore eliminates “first pass” liver metabolism and also avoids gastrointestinal discomfort. In a third aspect, the invention provides a method of administering nalmefene which results in a relatively gradual release of nalmefene over time when administered enterally so as to avoid large peaks in serum nalmefene concentration after per os administration.

The pharmacological and clinical advantages provided by these methods can only be achieved using the opioid antagonist nalmefene. As discussed above, nalmefene alone, in stark distinction from other opioid antagonists such as naloxone and naltrexone, has unique binding affinities for opioid-receptor subtypes, namely mu-receptors and kappa-receptors. The unique binding profile of nalmefene allows for preferred blocking at kappa-receptors relative to mu-receptors, such that dopamine release will tend to be less inhibited due to actions at kappa-receptors than would be the case with equivalent blocking at mu-receptors by other opioid antagonists such as naloxone or naltrexone.

### DETAILED DESCRIPTION OF THE INVENTION

Humans addicted to opioid agonist analgesics, such as buprenorphine, codeine, fentanyl, heroin, meperidine, methadone, morphine, opium, oxycodone, sufentanyl, and many other drugs classified as opioid narcotics, have a very difficult time abstaining from self-administering these analgesics, especially after detoxification and during the process associated with detoxification that is generally known as “withdrawal.” The present invention fulfills a long-awaited need to aid such humans so that addiction treatment for chemical dependencies on opioid agonist analgesics is greatly enhanced.

The invention encompasses a variety of methods for administering nalmefene that produce relatively constant release of nalmefene into the bloodstream of a human for a relatively prolonged or sustainable time. Thus, the serum concentration of nalmefene is less likely to have significant peaks and troughs over time as seen in association with intravenous bolus injections of nalmefene, or per os administration of nalmefene in a non-sustained release form.

The invention further provides for a practical way of accomplishing the above stated ends. For example, nalmefene can, and has been, administered by constant intravenous infusion in a post-surgical setting or following opioid overdose. However, this method has not been used as a method for addiction treatment. Further, intravenous infusion is cumbersome and not at all practical in ambulatory humans, especially those prone to opioid agonist analgesic addiction.

By stark contrast to a constant intravenous infusion of nalmefene, the present invention allows for nalmefene to be constantly absorbed into the bloodstream by way of very small capillaries found within living human tissue at prescribed constant rates, such as by diffusion through skin with transdermal delivery, by diffusion through fat with subcutaneous delivery—either by surgical implantation or needle injection into fatty tissue, by gradual absorption through the gastrointestinal tract in a sustained-release per os delivery method, by absorption through muscular tissue as with intramuscular injection, by absorption through mucosa as found in the gastrointestinal tract, or by diffusion through mucosal membranes as found in the sublingual area of the mouth or in nasal passages.

The following examples illustrate the present invention:

### EXAMPLE 1

There are a variety of transdermal delivery systems known in the art which deliver an array of medicinal agents in a sustained and constant fashion. Examples are Androderm® and Testoderm® systems that deliver testosterone, Alora™, Climara®, Estraderm® and Vivelle® systems which deliver estradiol, Catapres-TTS systems that deliver clonidine, Duragesic® systems which deliver fentanyl, Deponit®, Nitro Dur® and Transderm-Nitro® systems that deliver nitroglycerin, Habitrol®, Nicotrol® and ProStep® systems which deliver nicotine, and Transderm Scop® that delivers scopolamine.

The ideal steady state plasma concentration of nalmefene for blocking the effects of exogenously administered opioid agonist analgesics at mu-opioid receptors, while simultaneously allowing beta-endorphin to bind to and activate mu-opioid receptors, and effectively blocking dynorphins at kappa-receptors, in humans addicted to opioid agonist analgesics, is from about 1 to, about 3.7 ng/ml, most preferably between about 1.25 and about 2.5 ng-ml, such as 2.15 ng/ml. For a 70-kilogram (kg) adult (but not elderly) human, a sustained steady state plasma concentration for nalmefene of 2.0 ng/ml can be achieved by a transdermal delivery system in the following way.

Assuming an elimination constant (a.k.a. Kₑ) for nalmefene of 0.0642 hr⁻¹ (which is a value for Kₑ that is furnished by a distributor of nalmefene), and assuming an average volume of distribution (a.k.a. V_{d}) for nalmefene of 8.6 liters/kg (which has been shown to be an approximate average V_{d} in non-elderly adult humans for nalmefene), a target serum concentration in a 70 kg adult of 2.0 ng/ml nalmefene can be maintained by administering parenteral nalmefene, as administered transdermally, at an input rate of approximately 1.8 to 2.0 mg per day.

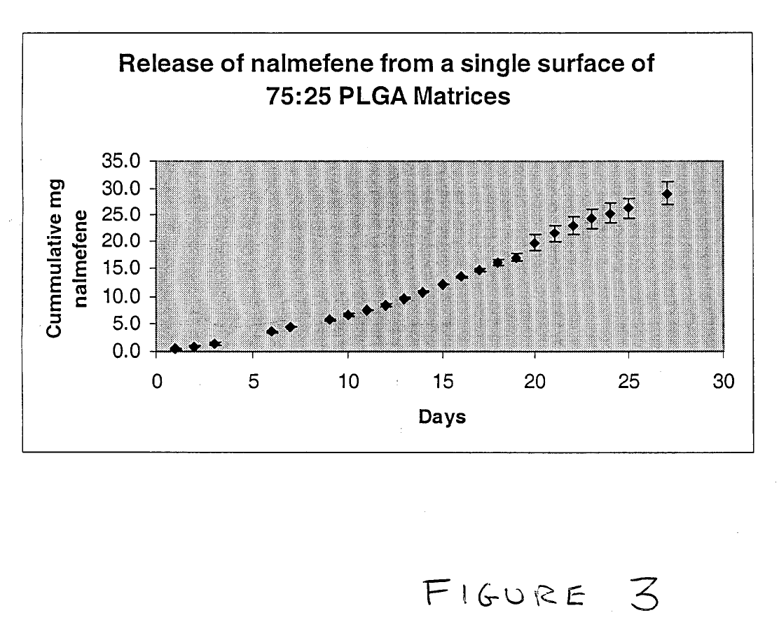

2.0 mg/day nalmefene can be effectively administered transdermally by constructing a transdermal delivery system described as follows: FIG. 1 illustrates a transdermal delivery system as taught by the present invention. The system is embodied in a transdermal patch, generally designated 10, comprising drug reservoir 12 which includes a matrix 14 having nalmefene base and PEGML dispersed therethrough. The reservoir 12 is covered by a impermeable backing layer 16 which is sized slightly larger in circumference than the reservoir. An adhesive overlay 18 is provided for adhering the patch to the surface of the patient's skin. The overlay is separated from the reservoir 12 by the peripheral portion 20 of the backing layer 16 surrounding the reservoir 12. This is required to prevent adverse reactions between the PEGML dispersed in the reservoir and the adhesive supported on the overlay. The patch 10 further includes an adhesive release liner 22 which is removed by the patient or clinician just prior to attaching the patch to the skin.

A number of different materials are suitable for forming the matrix 14. However, due to the solubility characteristics of PEGML, the matrix is preferably formed from an anhydrous material such as natural or synthetic rubbers other polymeric materials, thickened mineral oil or petroleum jelly, when PEGML is used as the flux enhancing compound. In the illustrated embodiment, the matrix is formed from an ethylene vinylacetate copolymer preferably having an vinylacetate content of from about 28% to about 60%.

The nalmefene is dispersed through the matrix at a concentration in excess of saturation, with the amount in excess of saturation being determined based on the intended useful life of the patch. Accordingly, the typical concentration of nalmefene in the reservoir is in the range of from about 10% to about 35% by weight. The PEGML is dispersed through the matrix at a concentrations below saturation and preferably between the range of activity of from about 0.25 to about 0.60. Thus, the reservoir typically contains from about 25% to about 60% PEGML by weight. Where various PEGML compositions having different average molecular weights of the PEG component can be utilized, a composition comprising PEG (200-400) ML is preferred.

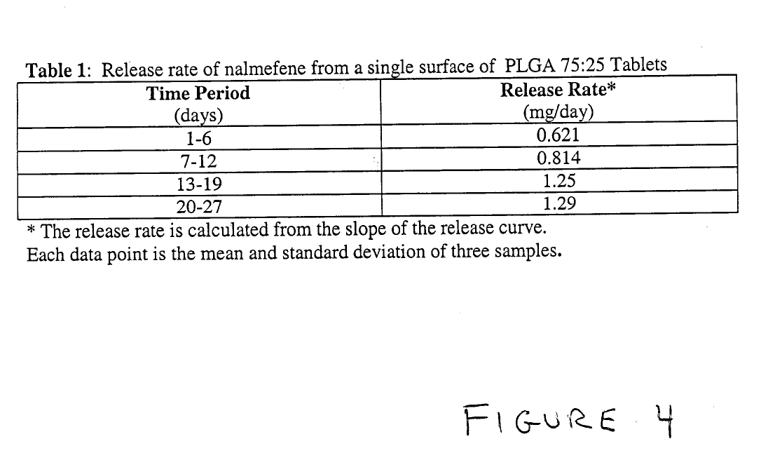

FIG. 2 illustrates a second embodiment of the transdermal patch. As shown in FIG. 2, the patch, generally designated 100, comprises a laminated reservoir 110 including layers 112 and 114. Layer 112 comprises a drug/flux enhancer reservoir substantially as described with respect to the reservoir 12 of FIG. 1. Thus, layer 112 includes as ethylenevinyl acetate matrix 116 having nalmefene and PEGML dispersed therethrough. Layer 114 includes a PEGML reservoir that utilizes essentially the same matrix material 118 as that found in layer 112. The matrix 118 has PEGML dispersed therethrough but is substantially free of any undissolved nalmefene.

The patch 110 further includes a semi-permeable membrane 120 between layers 112 and 114 which controls the release of PEGML from layer 114 into layer 112 and from layer 112 into the skin. The membrane 120 may be formed from any pharmaceutically acceptable material having low permeability to PEGML, and in the preferred embodiment the membrane is formed from ethylene-vinyl acetate copolymer having a lower vinyl acetate content than the matrix.

The advantage of the FIG. 2 embodiment is that the nalmefene is concentrated in layer 112 near the surface of the skin, rather than throughout the entire reservoir as is the case with reservoir 12 in FIG. 1. This permits reduced loading of nalmefene in the patch, while at the same time providing for a sufficient PEGML reservoir for the intended life of the delivery system.

The patch 100 further includes an impermeable backing layer 122 superimposed over the reservoir 110 and an adhesive overlay 124 as described above with respect to the FIG. 1 embodiment Also included is a release liner 126 that is removed just prior to attaching the patch to the skin.

To provide the desired plasma concentration of 2.0 ug/ml as described above, the patch is constructed, in one example, according to the FIG. 1 embodiment. The matrix is formulated with weight percentages of nalmefene and PEGML to provide an input rate of nalmefene of 20 ug/cm²/hr. A patch having this input rate is dimensioned to present approximately 4.2 cm² of reservoir surface area in contact with the skin. Typically, the patch is configured to be substantially square-shaped, though it may be round, oval or of another shape having a similar area, and in a square formation measures approximately 2.05 cm×2.05 cm. In another example, the patch is formulated to deliver an input rate of 3.5 ug/cm² of nalmefene per hour. To provide the required plasma level, the size of the patch must be substantially larger. A patch of this type is dimensioned to present 23.8 cm² of reservoir surface area in contact with the skin. A circular embodiment of this patch is configured as a circle having a radius of approximately 2.8 cm, and it is significantly larger than the previously described example. With either of these examples, steady state nalmefene concentrations can be reached faster by giving a loading dose of nalmefene, e.g. by intravenous bolus as in rapid opioid detoxification under anesthesia. Thus, the present invention is an extension of U.S. Pat. No. 5,783,583, which describes loading a human with nalmefene under anesthesia, then following the loading dose with a constant delivery of nalmefene.

To maintain a steady state plasma concentration of nalmefene of 2.15 ng/ml in a 80 kg human being, assuming a V_{d} of 8.6 L/kg and a Ke of 0.0642 hr⁻¹, a patch delivering an amount of nalmefene of approximately 2.3 mg per 24 hour period can be formulated as having an area of about 9.6 cm² if the percent by weight of nalmefene and flux enhancing compound is formulated to yield an input rate of nalmefene of about 10 ug/cm²/hr. An embodiment of such a patch could be a circular patch with a diameter of about 3.5 cm.

As the foregoing demonstrates, the size and input rate of a prescribed series of transdermal patches can be individually altered to provide transdermal dosages of nalmefene consistent with the present invention. Alterations in flux enhancers and other materials making up the transdermal patch are likewise applicable to the present invention. Thus, upregulation of opioid receptors, stimulation of endogenous beta-endorphin release, and optimal blocking effects at both mu- and kappa-opioid receptors by nalmefene, which all serve to optimally regulate dopamine release, can be accomplished without it being necessary for the patient to return to the clinic daily over the extended term of a nalmefene maintenance program.

Additional advantages result from the continuous and sustained nature of transdermal delivery of nalmefene. Because the drug becomes absorbed into the dermis, removing a transdermal patch does not instantaneously stop drug administration. The lag between the time the patch is removed and the time the drug ceases to be absorbed into the bloodstream is an effective tool against the compulsive behavior that is typical of opioid addicted humans who seek immediate gratification from their actions. If an addicted human wanted to stop nalmefene delivery in order to experience the effects of exogenous opioid agonist analgesics, this would have to be planned out in advance by removing the transdermal delivery system some time ahead of the anticipated drug use. Thus, impulsive actions on the part of the addict would not result in immediate results. Such a lapse, in many instances, is sufficient to deter the addicted human from impulsively discontinuing nalmefene therapy. In addition, the removal of the patch by the patient is quite apparent to the support person or clinician monitoring the patient, thus making the process of monitoring easier and more effective.

It may be the case that the dosage schedule over the course of a nalmefene maintenance protocol will have to be tailored for each individual patient. The transdermal delivery system of the present invention is ideally suited for individualized dosage regimens since the size, delivery rate and number of patches can be readily designed to meet the needs of a particular patient. Variations among patients include mass (weight in kilograms), volumes of distribution, and the particular state of opioid receptor regulation at a given time.

The transdermal delivery of nalmefene in an appropriate dosage negates the effects of exogenously administered opioid agonist analgesics while maintaining the effects of the natural opioid endorphin system to the greatest degree possible. The constant delivery of nalmefene results in relatively constant serum concentrations, so as not to result in high peaks of nalmefene concentration as occurs following a bolus administration of the drug. This is especially important in treating addiction to opioid agonist analgesics, because if a high peak concentration of nalmefene is reached after each bolus, the concentration of nalmefene at mu-opioid receptors may become high enough to block not only exogenous opioid agonist analgesics, but naturally occurring endorphins as well. This would be expected to result in dysphoria or other unpleasant effects. Such unpleasant effects, if repeatedly associated with being administered nalmefene, may result in the development of an aversion to being administered the drug. This dissuades the human from being compliant with a prescribed regimen of nalmefene administration. Humans addicted to opioid agonist analgesics are notoriously unreliable in following a regimen of self-administer medications per os. Thus, transdermal delivery of an antogonist can provide important advantages with respect of patient compliance, since addicted humans will exhibit a much higher compliance rate for the full term of the nalmefene maintenance protocol.

While it may be necessary for patients to periodically replace a number of transdermal patches to complete a nalmefene maintenance protocol, this can be accomplished under the supervision of a support person designated to assist in, and to monitor, the treatment of the addicted human. Monitoring can be facilitated by placing the transdermal patch on the surface of the patient's skin and marking the edge of the impermeable backing layer and a corresponding portion of the skin surface in one or more locations with indelible marker. Marking the patch and the skin in this manner registers the patch with the skin, such that if the addicted patient removed the patch it would be difficult for him or her to replace it with the patch in the exact orientation prior to removal. Using this method, addicts can easily be monitored since the support person or a clinician can readily determine if the patch had been left in place.

### EXAMPLE 2

Another way of delivering nalmefene both enterally in small incremental doses (as with normal swallowing) and parenterally (due to absorption sublingually) over a relatively prolonged period is to formulate a chewing gum preparation, such as nalmefene polacrilex, in a fashion somewhat resembling nicotine polacrilex which is marketed as Nicorette gum by SmithKline Beecham.

By formulating a nalmefene polacrilex gum with a given mass-unit of nalmefene per individual unit of chewing gum, the prescribed number of units of chewing gum can be administered to a human per over a prescribed amount of time to yield the preferred serum concentration of nalmefene, the prescribed number of units of gum per time-unit depending upon the lean body mass of a particular human.

### Example 3

Sustained administration of nalmefene may also be accomplished by surgically implanting an osmotic pump. The Alza Corporation manufactures osmotic pumps; one example is the surgically implantable ALZET® Osmotic pump, and another example is the OSMET osmotic pump for rectal administration. Both are capable of delivering nalmefene within the scope of the present invention.

For example, if the desired parenteral input rate into a human is 2.4 mg/day, then using ALZET® Osmotic Pump model #2ML 1 that delivers liquid at a rate of 10 microliters (ul) per hour, or 240 ul/day, if the concentration of nalmefene is 10 mg/milliliter (10 mg/cc), the desired input rate can be achieved. To thwart local tissue immunological reactions and pump “encapsulation,” a small dose of triamcinolone may be included in the osmotic pump for release to local tissue surrounding the implanted pump. In order to avoid subjective discomfort due to this foreign object being implanted subcutaneously, a pharmacologically compatible local anesthetic may also be included within the pump.

The particular osmotic pump embodied herein is described for illustrative purposes only, and is not intended to limit the scope of the present invention, which is consistent other osmotic pump release devices.

### EXAMPLE 4

Nalmefene may be prepared as nalmefene polistirex, in a fashion similar to the known preparation of dextromethorphan polistirex. Such a form of dextromethorphan polistirex is manufactured by Mediva Pharmaceuticals, Inc. in Fort Worth, Tex. and is marketed as Delsym®, which provides an extended release of dextromethorphan over approximately 12 hours.

The preferred dose of nalmefene polistirex is based on the lean body mass of the treated human. By formulating an elixer of nalmefene polistirex with a given mass-unit of nalmefene per volume-unit, the prescribed amount of nalmefene can be administered that results in the preferred serum concentration of nalmefene.

### EXAMPLE 5

There are a variety of intranasal delivery systems in the prior art that deliver various medicinal agents in a parenteral, non-intravenous fashion via absorption through the nasal mucosa. Examples are Atrovent® nasal spray that delivers ipatropium bromide, Flonase® nasal spray which delivers fluticasaone propionate, Stadol NS® which delivers butorphanol tartrate, Beconase AQ® nasal spray that delivers beclomethasone dipropionate monohydrate, Nicotrol®NS nasal spray which delivers nicotine, Miacalcin® nasal spray which delivers calcitonin-salmon, DDAVP® nasal spray which delivers desmopressin acetate, Nasacort® AQ nasal spray and Nasacort® nasal inhaler which deliver triamcinolone acetonide, Nascobal™ gel that delivers cyanocobalamin, and Astelin® nasal spray which delivers azelastine hydrochloride.

According to the present invention, nalmefene is prepared as a free base or in its salt form and incorporated into a pharmacologically suitable nasal carrier, in a manner known to those skilled in the art. The choice of suitable carrier will depend upon whether the route of administration is by nasal solution, nasal suspension, or nasal aerosol using a volatile propellant. Generally, water is used in formulating a preparation, and the pH of the preparation may be altered by any one of known pH adjusters, e.g. sodium hydroxide.

A tartrate, stearate or palmitate formulation of nalmefene may be used, or nalmefene may be in the form of nalmefene hydrochloride, and formulated such that 1 gram of active nalmefene is mixed with 80 ml of distilled water, then adjusted to a pH of approximately 7.4 with dilute sodium hydroxide, and then isotonic saline is added along with a suitable preservative and antibacterial agent, to yield a total volume of 100 ml. This yields a nalmefene solution with a concentration of 10 mg/ml nalmefene. This final solution is passed through a 0.2 micron Millipor filter to remove bacteria and other undesired particles. The filtered solution is then placed aseptically into a container to which is then attached a metered dosino mechanism which allows approximately 0.1 ml to be delivered in each spray. An example of such a metered dosing system in found with the commercially marketed Stadol NS® (Bristol-Myers Squibb Co.). One spray to one nostril may be expected to yield a blood serum concentration of approximately 1 ng/ml 30-90 minutes after administration. Because the serum half life of nalmefene is approximately 10.8 hours, 5 consecutive 1 mg doses approximately 11 hours apart will result in steady state serum concentrations of nalmefene of approximately 1 ng/ml. Alternatively, the human may be given a loading dose of intravenous nalmefene and then transferred to a nasal administration regimen. A practical dosing schedule for nalmefene may be 1.5 mg intranasally every 12 hours. To facilitate this dosing regimen, 1.5 g of nalmefene may be substituted for the 1.0 mg previously described for preparation of a nasal solution, thus yielding a final concentration of 15 mg/ml, which can be administered in 0.1 ml increments.

In addition, permeation enhancers may be added to the nasal solution to increase input through the nasal mucosa Palmitoyl and stearyl components of lysophosphatidylcholine in 0.5% concentration, are examples of mucosal permeation enhancers. Lysolecithin is a very potent mucosal permeation enhancer.

It is understood that these examples are for illustrative purposes only and are not to be construed-as limiting the invention in spirit or scope.

### EXAMPLE 6

Propellant-based aerosol systems for immediate non-intravenous parenteral delivery of a medicinal agent through oral mucosa sublingually are known in the art. For example, Nitrolingual® spray delivers nitroglycerin, which circumvents first-pass liver metabolism. This formulation utilizes dichlorodifluoromethane and dichlorotetrafluoroethan as propellants. Like propellants can be used in an aerosolized formulation of nalmefene which would be concentrated to deliver a dosing regimen similar to that described in Example 5. The manner of preparing a suitable formulation would be apparent to one skilled in the art in light of the present invention.

### EXAMPLE 7

There are a variety of depot preparations for subcutaneous or intramuscular injection which provide for sustainable delivery of medicinal agents at a relatively even rate. These may employ particular methods that vary from one depot system to another. Examples include Lupron Depot® systems that deliver leuprolide acetate, DepoProvera® that delivers medroxyprogesterone acetate and Zoladex® which delivers goserelin acetate.

The method for a sustainable releasing formulation of nalmefene, which is easily parenterally injected into subcutaneous or muscular tissue, may be as simple as preparing nalmefene in an oil base such as sterile peanut oil. More elaborate systems allow for a more controlled rate of release such that steady state serum concentrations of nalmefene are more constant. These systems may entail putting microencapsulated particles of nalmefene into a suspension that can then be delivered through a percutaneous needle. For instance, a polymer of a natural compound or compounds, such as a polymer or copolymer which includes biodegradable poly-lactic and poly-glycolic acids, polylactic acid, polyglucolic acid, polylactones, or any of a number of biodegradable non-toxic polymers, is used to encase or encapsulate particles of nalmefene. Poly(L(+)-lactic acid) and DL-lactic acid have been used in the prior art for sustained release drug formulations. These “microcapsules” are then suspended in a carrier solution. After being injected parenterally, preferably by subcutaneous route, the microcapsules break down over time thereby releasing active nalmefene for capillary absorption into the bloodstream. By varying the ratio of polymers in a copolymer, or by using different polymers or coploymers in a given suspension, and by varying the size of the microcapsules, the nalmefene can be released “in waves” from the suspension. In light of the present invention, one skilled in the art would be able to formulate a particle size of nalmefene, a microcapsule of the required size and composition, such that nalmefene would be released in a sustainable fashion while yielding relatively constant steady state serum concentrations of nalmefene consistent with the present invention.

As noted above, inclusion of a steroidal anti-inflammatory agent, e.g. triamcinolone, and a pharmacologically compatible local anesthetic, may provide the added benefits of greater comfort to the human administered the composition, as well as providing a means to decrease local tissue inflammatory responses which may cause induration or pain at the injection site.

### EXAMPLE 8

There exists in the prior art a variety of surgically implantable delivery systems, one such example is the Norplant® system that delivers levonorgestrel. Grossman et al in U.S. Pat. No. 5,633,000 ('000) teach a subcutaneous implant comprising a poly(ethylene-vinyl acetate) matrix in which active drug is embedded. Nalmefene is not an equivalent drug to the active drug in claimed in '000. Further, the preparation of '000 is alleged to be “non-inflammatory, biocompatible and non-biodegradable,” which therefore results in a prolonged, controlled release of active drug with “near zero-order” kinetics.

### EXAMPLE 9

The prior art shows many sustained- or controlled-release tablets and capsules for per os administration. The oral controlled-release system is often made of polymers that release active drug by diffusion, bio-erosion, or swelling due to increased osmotic pressure generated in the gastrointestinal tract. Diffusion controlled systems contain a reservoir, matrix and porous membrane.

One method for producing sustained delivery of oral medications is to encapsulate active drug with slowly dissolving polymeric materials. The rate of release of active drug is influenced by the thickness and the dissolution rate of the particular polymeric coat of the active drug. By varying the thickness and dissolution rates of coated drug particles in a particular preparation, active drug will be released at different predetermined times. This is generally known as microencapsulation.

Another method for producing sustained or controlled delivery of orally administered drug is the matrix dissolution method. A means for preparing a drug-polymer matrix is “congealing” where the drug is mixed with polymeric substances or waxes. A specific method of congealing is known as “spray-congealing.” Another means for matrix preparation is the aqueous-dispersion method. In this method, the drug-polymer mixture is sprayed or placed in water and then collected.

### EXAMPLE 10

A hybrid of oral administration and osmotic pump delivery is the OROS® system developed by Alza Corporation. In this method, a non-digestible capsule is made of a semi-permeable membrane. Within the confines of this membrane is an osmotic core containing the active drug. As water passes through the semipermeable membrane due to an osmotic gradient, the water tends to push the active drug through an orifice in the capsule. This provides for constant delivery of active drug as the capsule passes through the gastrointestinal system. An example of this system is Acutrim®, which releases phenylpropanolamine in a sustained dose that causes appetite suppression but which produces little, if any, adrenergic-like side effects which typically accompany phenylpropanolamine administration by less controlled bolus administration.

Such non-digestible per os administration of nalmefene may provide a relatively easy solution to sustained action and controlled release of nalmefene consistent with the present invention.

### EXAMPLE 11

Bupropion is co-administered with any of the above examples of nalmefene administration. When administered orally, a sustained release of bupropion is preferred, such as Wellbutrin®SR or Zyban®. Bupropion may be administered in a variety of delivery systems as previously described for nalmefene. A preparation combining the two active drugs nalmefene and bupropion may also be formulated. However, the simple co-administration of an orally administered sustained release tablet of bupropion HCL with nalmefene is suitable for optimizing dopamine release in the central nervous system in the setting of partially blocked mu-opioid receptors.

The invention also provides a method for sterilizing the above-described sustained-release delivery systems. The method comprises exposing a system to sufficient xray radiation to destroy any contaminating microorganisms, without causing any harmful effects to either the active ingredients contained in the sustained-release delivery system or to the composition of the materials comprising the system.

While preferred embodiments have been shown and described, various modifications and substitutions may be made without departing from the spirit and scope of the invention. Accordingly, it is to be understood that the present invention has been described by way of example and not by limitation.

### New Material

Hydrogels are polymer networks that swell in water without immediately dissolving. They typically possess good biocompatibility because of low interfacial tension with surrounding biological fluids and the rubbery characteristic that minimizes mechanical and frictional irritation to biological tissue with which they may come in contact. Hydrogels may be made of biodegradable materials and polymers. However, “low-molecular weight water-soluble drugs often permeate hydrogels at too high a rate to be useful” in a non-membrane form [Drug Delivery Systems, p.287, Vasante Ranade and Mannfred Hollinger, ISBN 0-84938542-3].

A drug such as nalmefene may be held within a hydrogel composition. Hydrogels may release such a drug by one of several mechanisms. If the hydrogel is a biodegradable polymer, the drug may be released as the polymer is broken down, such as by hydrolysis. Such hydrogels release a drug by erosion of the drug delivery device structure, for example erosion of a polymer network. A second way that the hydrogel may release a drug is that the hydrogel takes in water and swells, as a result of this the network mesh of polymers of which it is made is stretched. Such stretching increases the distance between individual polymers making up the hydrogel network. As this occurs, drug molecules (e.g. nalmefene) may then slip through the individual polymers and diffuse out of the hydrogel network at a rate controlled by the drug's concentration gradient and the distance between individual polymers (which is proportional to swelling of the hydrogel).

Additional factors that effect biodegradation or erosion of the hydrogel in biological systems (in vivo) are the effects of enzymes that are in contact with the hydrogel, and the local inflammatory response that occurs when a foreign body (e.g., a hydrogel) is in contact with biological tissue. For instance, it is well known that many drug delivery devices that are administered to the subcutaneous tissues of an animal or human cause an inflammatory reaction that may, 1) result in phagocytosis or digestion by immunological cells, or 2) result in a deposition of fibrous tissue around the foreign body such that the foreign body will be “walled off” from the immediate subcutaneous tissue outside of the area of the fibrous wall containing the hydrogel. Either of these responses would significantly affect the release rate of drug (e.g. nalmefene) from the hydrogel or foreign body. Thus, incorporation of an anti-inflammatory drug, such as triamcinolone, would directly affect the release rate of the drug from the hydrogel. This affect of anti-inflammatory drugs on drug release rate has not been appreciated by those skilled in the prior art.

Further, because hydrogels typically swell, this may produce hydrostatic pressure against surrounding biological tissue. For example, if a hydrogel is administered to the subcutaneous space in a human, the swelling could cause pressure on the nerves in the subcutaneous tissue resulting in pain. Expected pain would naturally dissuade a human from agreeing to have the hydrogel administered in the subcutaneous space. This also has not been appreciated by those skilled in the art. The inclusion of a local anesthetic drug such as procaine or 2-chloroprocaine would alleviate or prevent the pain as the local anesthetic would be released from the hydrogel and bathe surrounding nerves in the subcutaneous tissue.

It can be important to deliver an active drug such as nalmefene in its hydrochloride salt form because administration of the pure base form of the drug can result in caustic changes in pH in the area immediate to the hydrogel, which can result in irritation and further pain. In addition, the hydrochloride salt form of nalmefene and many other drugs are much more chemically stable than the corresponding pure base forms. Thus, if the goal is to deliver a specific active drug (e.g. nalmefene) into the bloodstream at a given optimally therapeutic rate in order to result in an optimum blood concentration of the drug, the chemical stability of the drug becomes of paramount importance.

An comprehensive review of biodegradable injectable drug delivery systems is given by reference in Journal of Controlled Drug Release 80 (2002), pages 9-20 —“Biodegradable injectable in-situ forming drug delivery systems” authored by A. Hatefi and B. Amsden. The present invention differs from those described by Hatefi and Amsden in that the present invention does not form in situ after injection or administration in vivo, but rather is formed ex situ or in vitro. In other words, the present invention is manufactured to completion outside of a living animal or human and maintains its physical characteristics immediately after administration by injection.

The present invention allows for a desirable rate-controlled release of water-soluble molecules from a semi-solid (“semi-sol”) that can be readily injected through a hypodermic needle into subcutaneous tissue. This semi-sol shares some characteristics described above for a hydrogel. It is biodegradable, non-toxic in vivo, and is made of a network of a copolymer. It holds its general shape and form in vivo. However, unlike other drug delivery devices in the prior art that may be injected through a needle and then become a harder discrete form in vivo, the present invention is not based on a solvent diffusing away after subcutaneous administration, and is not based on temperature differences between room temperature and in vivo body temperature, an will maintain its ex vivo or in vitro general form in vivo.

Though the model drug used herein is nalmefene hydrochloride, the present invention in no way is meant to be limited to this particular drug, but rather can have embodiments that encompass many other water-soluble small molecules. When nalmefene is embodied as an active drug of the invention, it may be used preferentially to treat opioid dependence, alcoholism, cocaine dependence, compulsive behaviors such as gambling and sexual addictions, as well as treating HIV-infected individuals to diminish HIV virus activation and replication.

### EXAMPLE 12

A novels moldable drug delivery formulation with putty-like characteristics that releases nalmefene at a desired rate at 37 degrees Celsius and pH of 7.4 is described. The nalmefene-containing formulation may be injected through a typical hypodermic needle to make subcutaneous administration through an 18-gauge needle relatively simple.

Poly(DL-lactide-co-glycolide) copolymers (“PLGA”) with a ratio of lactide to glycolide of 75:25 having an inherent viscosity of 0.69 dL/g in CHCL₃ at 30 degrees Celsius were purchased from Birmingham Polymers, Inc. (Birmingham, Ala.). 2 grams of the PLGA were dissolved in 1 milliliter (“ml”) of the solvent acetone (“solvent 1”). 2 ml of the plasticizing agent triacetin is added to the PLGA-acetone mixture while being stirred. Nalmefene hydrochloride (“HCL”) was purchased from Mallinckrodt, Inc. (St. Louis, Mo.). The nalmefene is dissolved in a solvent (“solvent 2”), examples of which are distilled water and pure ethanol. Low heat, 40-60 degrees Celsius may be used to enhance solubility of nalmefene in the solvent. In one method, the 800 milligrams (“mg”) nalmefene is dissolved in 2 ml (2 grams) of distilled water and 0.575 g PEG 300 at 50 degrees Celsius (“C”). “PEG” is polyethylene glycol, and 300 refers to PEG have specific characteristics. The nalmefene-solvent 2-PEG mixture is then added to the PLGA-solvent1-triacetin mixture with stirring and enough heat to optimally catalyze the mixing or blending of the two mixtures into a new combined mixture. In this instance, temperature in the range of 60-80 degrees C. is used. After stirring to a relatively uniform combined mixture or blend, the combined mixture is placed in a vacuum oven at 60 degrees C. for approximately one hour. The negative pressure within the vacuum oven is adjusted so as to allow solvent 1 and solvent 2 to evaporate without having the combined mixture boil or overflow its container. Alternatively, the combined mixture may be subject to a roto-vaporizor. In this instance, a vacuum oven is utilized as described. The combined mixture is then spread out relatively uniformly on a non-stick surface such as a Teflon® plate and placed once again in the vacuum oven for 30 minutes or more. The soft moldable putty-like material containing nalmefene HCL, triacetin, PEG 300 and PLGA is again manually mixed, stirred or kneaded into a uniform consistency. It is sticky when removed from the oven, but upon cooling the semi-sol delivery blend is easier to manually manipulate and work with. It may be cooled in a water bath. At room temperature is readily moldable and has a putty-like consistency. This moldable semi-sol may then be molded into a tablet configuration, injection molded, or in may be inserted into a syringe and injected through a hypodermic needle. The viscosity of the putty, or its ability to be easily injected through a needle of a specified diameter and length, can be readily manipulated by changing the relative amounts of PLGA, triacetin and PEG. In this instance PEG 300 is used, however PEG 400 or other PEG's may be used.

A moldable semi-sol prepared as just described was put with a phosphate buffer having a pH of 7.4 into a glass vial. The glass vial was then put into a shaker bath at 37 degrees Celsius and release rate of nalmefene were monitored for 27 days. FIG. 3 shows the release rate of nalmefene from three samples. The data show that there is no initial burst of nalmefene and that the rate of release slowly increased over time. These characteristics of nalmefene release are very important when nalmefene is used to treat an opioid dependent human immediately after detoxification or withdrawal. This is because the gradual increase in release of nalmefene allows a relatively smaller amount of nalmefene to be administered soon after detoxification when the human subject is most sensitive to the adverse effects of nalmefene, and allows for a relatively larger, and therefore “more protective,” amount of nalmefene later on as the human subject adapts to not having mu-opioid receptors activated by an opioid agonist. This is expected to produce a much “smoother” time period in the days following detoxification when the nalmefene is administered, such as by subcutaneous injection of the semi-sol sustained deliver system.

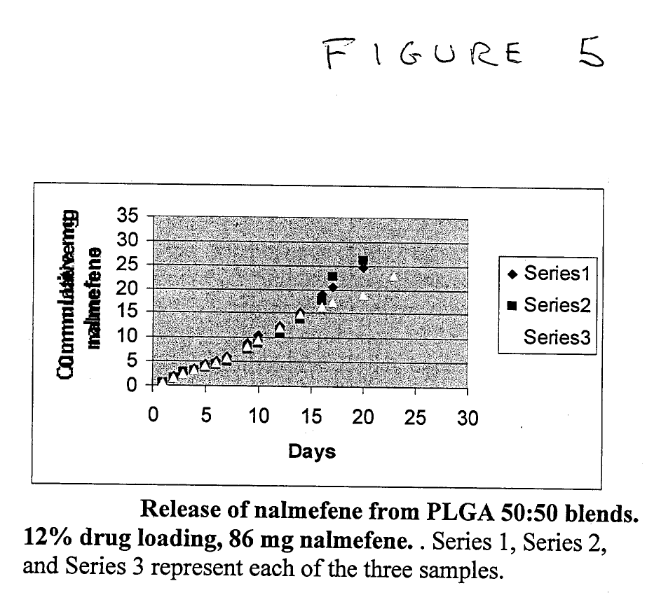

Since there was a gradual increase in nalmefene release over the 27 days, the data in FIG. 3 are not absolutely linear. Since the release rate was not absolutely linear, the data was analyzed at 6-day intervals. Since the putty-like semi-sol formed a thin disc in the glass vial (resembling a tidily wink at the bottom of the vial), the release was essentially “one-sided.” Nevertheless, the measured release rate was 0.6 mg/day for the first few days to a maximum of 1.29 mg/day at the near the 27^{th} day (see FIG. 4). The putty swelled with time at a gradual rate. In models described in the prior art, the release rate of a drug or molecule (e.g. nalmefene) in swelling hydrogels is presumed to be diffusion controlled and dependent upon the buffer into the hydrogel. The rate of release would be expected to decrease as the nalmefene became depleted and as the diffusional pathlength increased with swelling. Most unexpectedly, the opposite effect is seen in the present invention, i.e., the rate of release increases with swelling. Thus, the present invention appears to be associated with a new and novel way in which water-soluble molecules are released from a putty-like semi-sol delivery system that swells when in contact with water.

### EXAMPLE 13

The invention generally described above except that the plasticizing agent may be triethyl citrate instead of triacetin.

### EXAMPLE 14

A novels moldable drug delivery formulation with putty-like characteristics that releases buprenorphine at physiological conditions is described. The buprenorphine-containing formulation may be injected through a typical hypodermic needle to make subcutaneous administration through an 18-gauge needle relatively simple.

Poly(DL-lactide-co-glycolide) copolymers (“PLGA”) with a ratio of lactide to of 75:25 having an inherent viscosity of 0.69 dL/g in CHCL₃ at 30 degrees Celsius were purchased from Birmingham Polymers, Inc. (Birmingham, Ala.). 2 grams of the PLGA were dissolved in 1 milliliter (“ml”) of the solvent acetone (“solvent 1”). 2 ml of the plasticizing agent triethyl citrate is added to the PLGA-acetone mixture while being stirred. Buprenorphine HCl is dissolved in a suitable solvent (“solvent 2”) that evaporates at a temperature and pressure such that the solvent can be readily evaporated without physical breakdown of the buprenorphine core molecule. Low heat, 40-80 degrees Celsius may be used to enhance solubility of buprenorphine in the solvent. In one method, the 800 milligrams (“mg”) buprenorphine is dissolved in 2 ml (2 grams) of distilled water and 0.575 g PEG 400 at 50 degrees Celsius (“C”). The buprenorphine-solvent 2-PEG mixture is then added to the PLGA-solvent1-triethyl citrate mixture with stirring and enough heat to optimally catalyze the mixing or blending of the two mixtures into a new combined mixture. In this instance, temperature in the range of 50-80 degrees C. is used. After stirring to a relatively uniform combined mixture or blend, the combined mixture is placed in a vacuum oven at 60 degrees C. for approximately one hour. The negative pressure within the vacuum oven is adjusted so as to allow solvent 1 and solvent 2 to evaporate without having the combined mixture boil or overflow its container. Alternatively, the combined mixture may be subject to a rotovaporizor. In this instance, a vacuum oven is utilized as described. The combined mixture is then spread out relatively uniformly on a non-stick surface such as a Teflon® plate and placed once again in the vacuum oven for 30 minutes or more. The soft moldable putty-like material containing buprenorphine HCL, triethyl citrate, PEG 400 and PLGA is again manually mixed, stirred or kneaded into a uniform consistency. It is sticky when removed from the oven, but upon cooling the semi-sol delivery blend is easier to manually manipulate and work with. It may be uniformly blended at this stage as taffy is manufactured on a “taffy machine.” It may be cooled in a water bath. At room temperature is readily moldable and has a putty-like consistency. This moldable semi-sol may then be molded into a tablet configuration, injection molded, or in may be inserted into a syringe and injected through a hypodermic needle. The viscosity of the putty, or its ability to be easily injected through a needle of a specified diameter and length, can be readily manipulated by changing the relative amounts of PLGA, triacetin and PEG. In this instance PEG 400 is used, however other PEG's may be used.

A 2 milliliter volume of the putty-like semi-sol preparation containing buprenorphine is then put into a 3 cc syringe attached to an 18 gauge needle, the syringe plunger in added air is expelled from the syringe. The syringe containing the putty-like semi-sol preparation containing buprenorphine is then sealed in a package. The sealed package is then subject to enough gamma radiation to effective sterilize the sealed packet with its contents that include the syringe, syringe plunger and the putty-like semi-sol preparation containing buprenorphine. This may now be used for the subcutaneous administration of the sustained drug delivery device by the usual methods.

The characteristics of buprenorphine release from this invention are very important when buprenorphine is used to treat an opioid dependent human that has not been detoxified. This is because the gradual increase in release of buprenorphine allows a relatively smaller amount of buprenorphine to be administered soon after buprenorphine maintenance therapy is initiated when the human subject is most sensitive to the adverse effects of buprenorphine, and allows for a relatively larger, and therefore “more protective,” amount of buprenorphine later on as the human subject adapts to having mu-opioid receptors only partially activated by an partial mu-opioid agonist buprenorphine. This is expected to reduce adverse effects associated with buprenorphine during time period in the days following initiation of buprenorphine maintenance by way of subcutaneous injection of the semi-sol sustained deliver system.

### EXAMPLE 15

The invention generally described in the above examples above except that the active opioid drug is nalbuphine.

### EXAMPLE 16

A novels moldable drug delivery formulation with putty-like characteristics that releases nalmefene at in vitro conditions is described. The nalmefene-containing formulation may be injected through a typical hypodermic needle to make subcutaneous administration through an 18 gauge needle relatively simple.

Poly(DL-lactide-co-glycolide) copolymers (“PLGA”) with a ratio of lactide to glycolide of 75:25 are used. 2 grams of the PLGA were dissolved in 1 milliliter (“ml”) of the solvent acetone (“solvent 1”). 2 ml of the plasticizing agent triacetin is added to the PLGA acetone mixture while being stirred. Into the PLGA-solvent 1-triacetin mixture, 5 to 10 mg triamcinolone is added. Triamcinolone is commercially available in various pharmaceutical configurations, such as triamcinolone base, triamcinolone acetonide, triamcinolone diacetate, and so forth. A form of triamcinolone that is easily soluble in the PLGA-solvent 1-triacetin mixture used.

Nalmefene hydrochloride (“HCL”) was purchased from Mallinckrodt, Inc. (St. Louis, Mo.). The nalmefene is dissolved in a solvent (“solvent 2”), in this case 2 ml pure ethanol. Low heat, 40-60 degrees Celsius may be used to enhance solubility of nalmefene in the solvent. 800 milligrams (“mg”) nalmefene is dissolved in 2 ml (2 grams) of ethanol and 0.575 g PEG 400 at 50 degrees Celsius (“C”). The nalmefene-ethanol-PEG mixture is then added to the PLGA-solvent1-triacetin-triamcinolone mixture with stirring and enough heat to optimally catalyze the mixing or blending of the two mixtures into a new combined mixture. In this instance, temperature in the range of 60-80 degrees C. is used. After stirring to a relatively uniform combined mixture or blend, the combined mixture is placed in a vacuum oven at 60 degrees C. for approximately one hour. The negative pressure within the vacuum oven is adjusted so as to allow solvent 1 and solvent 2 to evaporate without having the combined mixture boil or overflow its container. Alternatively, the combined mixture may be subject to a roto-vaporizor. In this instance, a vacuum oven is utilized as described. The combined mixture is then spread out relatively uniformly on a non-stick surface such as a Teflon® plate and placed once again in the vacuum oven for 30 minutes or more. The soft moldable putty-like material containing nalmefene HCL, triamcinolone, triacetin, PEG 400 and PLGA is again manually mixed, stirred or kneaded into a uniform consistency. It is sticky when removed from the oven, but upon cooling the semi-sol delivery blend is easier to manually manipulate and work with. It may be cooled in a water bath. At room temperature is readily moldable and has a putty-like consistency. This moldable semi-sol may then be molded into a tablet configuration, injection molded, or in may be inserted into a syringe and injected through a hypodermic needle. The viscosity of the putty, or its ability to be easily injected through a needle of a specified diameter and length, can be readily manipulated by changing the relative amounts of PLGA, triacetin and PEG. In this instance PEG 400 is used, other PEG's may be used. Further, other PLGA's may be used such as PLGA 65:35, PLGA 60:40, PLGA 85:15, etc.

The added advantage of this embodiment is that the semi-sol putty will be more resistant to phagocytosis induced by a foreign body reaction, thus leaving the putty intact in vivo long enough for the nalmefene to diffuse out of the putty. After the greater portion of the nalmefene leaves the putty, the putty is then left for bio-erosion or biodegradation. If however, the putty is degraded so fast such that the greater portion of nalmefene is still within it upon erosion, then a significant drug burst (of nalmefene) can be expected. Thus, the triamcinolone is an important component of the invention for in vivo use, which contrasts to the in vitro data depicted in FIGS. 3 and 4. This effect of triamcinolone in preventing drug burst and allowing for a continued release rate of nalmefene that is not drastically accelerating, has not been appreciated in the prior art. Triamcinolone is the preferred steroidal anti-inflammatory drug because it tends to have a more local effect on the immediately surrounding tissue and less of a systemic effect throughout the body, as compared to some of the other steroidal anti-inflammatory drugs.

### EXAMPLE 17

The invention generally described in the above examples above except that an active water-soluble drug delivered in sustained release fashion with the characteristics described is a salt form of naltrexone such as naltrexone hydrochloride.

### EXAMPLE 18

The invention generally described in the above examples above except that an active water-soluble drug delivered in sustained release fashion with the characteristics described is a salt form of 6-methyl-naltrexone such as 6-methyl-naltrexone hydrochloride.

### EXAMPLE 19

The invention generally described in the above examples above except that an active water-soluble drug delivered in sustained release fashion with the characteristics described is a water-soluble salt form of 6-beta-naltrexol.

### EXAMPLE 20

The invention generally described in the above examples above except that an active water-soluble drug delivered in sustained release fashion with the characteristics described is a kappa-opioid receptor selective antagonist such as nor-binaltorphimine (“nor-BNI”).

### EXAMPLE 21

The invention generally described in the above examples above except that an active water-soluble drug delivered in sustained release fashion with the characteristics described is a salt form of a kappa-opioid receptor selective agonist such as U50,488 or enadoline.

### EXAMPLE 22

The invention generally described in the above examples above except that an active water-soluble drug delivered in sustained release fashion with the characteristics described is a salt form of a delta-opioid agonist such as benzamide, SNC-80 or deltorphin.

### EXAMPLE 23

Poly(DL-lactide-co-glycolide) copolymers (“PLGA”) with a ratio of lactide to glycolide of 50:50 having an inherent viscosity of 0.59 dL/g in HFIP at 30 degrees Celsius were purchased from Birmingham Polymers, Inc. (Birmingham, Ala.). 2 grams of the PLGA were dissolved in 2 milliliter (“ml”) of the solvent acetone (“solvent 1”). 1 ml of the plasticizing agent triacetin is added to the PLGA-acetone mixture while being stirred. Nalmefene hydrochloride (“HCL”) was purchased from Mallinckrodt, Inc. (St. Louis, Mo.). The nalmefene is dissolved in a solvent (“solvent 2”), examples of which are distilled water and pure ethanol. Low heat, 40 degrees Celsius may be used to enhance solubility of nalmefene in the solvent. 500 milligrams (“mg”) nalmefene is dissolved in 1 ml (1 grams) of distilled water and 0.420 g PEG 300 at 40 degrees Celsius (“C”). The nalmefene-solvent 2-PEG mixture is then added to the PLGA-solvent1-triacetin mixture with stirring and enough heat to optimally catalyze the mixing or blending of the two mixtures into a new combined mixture. In this instance, temperature in the range of 45-65 degrees C. is used. After stirring to a relatively uniform combined mixture or blend, the combined mixture is placed in a vacuum oven at 60 degrees C. for approximately one hour. The negative pressure within the vacuum oven is adjusted so as to allow solvent 1 and solvent 2 to evaporate without having the combined mixture boil or overflow its container. Alternatively, the combined mixture may be subject to a roto-vaporizor. In this instance, a vacuum oven is utilized as described. The combined mixture is then spread out relatively uniformly on a non-stick surface such as a Teflon® plate and placed once again in the vacuum oven for 30 minutes or more. The soft moldable putty-like material containing nalmefene HCL, triacetin, PEG 300 and PLGA is again manually mixed, stirred or kneaded into a uniform consistency. At room temperature it is readily moldable and has a putty-like consistency without the need for additional cooling. This moldable semi-sol is molded into a tablet configuration weighing out 0.700 g of the final putty and pressing it into a circular mold with the dimension of 15 mm diameter by 3 mm depth. Alternatively, this putting is easily injection molded to the shape and size desired. The nalmefene drug loading for each tablet is 12%.

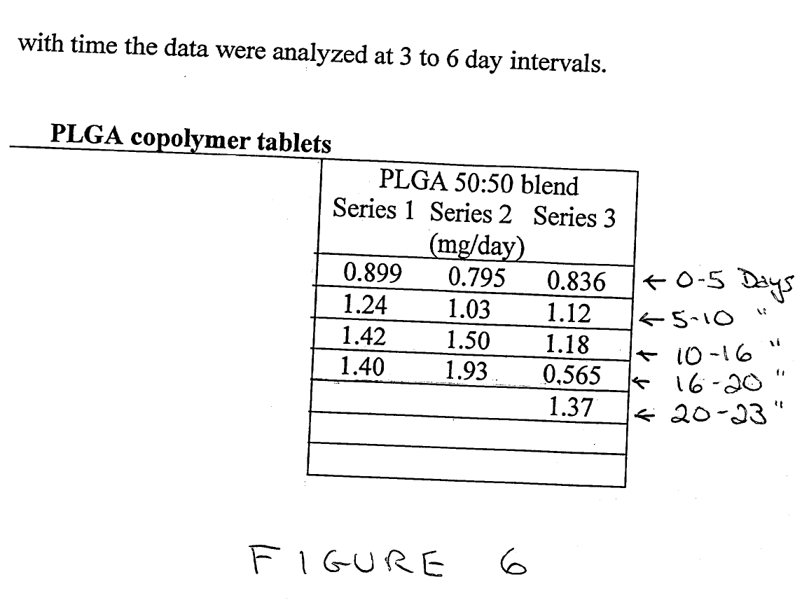

A moldable semi-sol tablet prepared as just described was put with 10 ml phosphate buffer having a pH of 7.4 into a 20 ml scintillation via into which glass wool was previously laid. After three days, the glass wool was replaced with a wire mesh, and on day 6 with Teflon®. The buffer was replaced with 10 ml of fresh solution after each measurement of the concentration of nalmefene. The measured nalmefene concentration was measured against a standard curve using know concentrations of nalmefene. The cumulative weight of nalmefene was plotted versus time. The nalmefene release rate was calculated by determining the slope of the curve at a given time interval. FIG. 5 depicts a graph of the release rate of nalmefene from three samples of the PLGA 50:50 blended putty containing 86 mg of nalmefene over time. FIG. 6 is data on nalmefene release rates at time intervals from 0-5 days, 5-10 days, 10-16 days, 16-20 days and 20-23 days. The data show that there is no initial burst of nalmefene and that the rate of release slowly increased over time. These characteristics of nalmefene release are very important when nalmefene is used to treat an opioid dependent human immediately after detoxification or withdrawal. This is because the gradual increase in release of nalmefene allows a relatively smaller amount of nalmefene to be administered soon after detoxification when the human subject is most sensitive to the adverse effects of nalmefene, and allows for a relatively larger, and therefore “more protective,” amount of nalmefene later on as the human subject adapts to not having muopioid receptors activated by an opioid agonist. This is expected to produce a much “smoother” time period in the days following detoxification when the nalmefene is administered, such as by subcutaneous injection of the semi-sol sustained deliver system.

The putty swelled with time at a gradual rate. The PLGA 50:50 putty blend swelled to only, taking in a maximum amount of water from the buffer solution by day 10, and the amount of swelling was only 60% of the original volume. This represents a maximum increase in size of 1.6 times, a volume that would be well tolerated by gradual swelling when administered into subcutaneous tissue. The tablet began to disintegrate after several weeks. This is desirable because the biodegradable implant is intended for complete absorption in vivo, thus removal of a drug delivery device shall not be required after the nalmefene is released.

Using various blends, it was determined by experimentation that degradation occurred with PLGA 50:50 putty blends after several weeks, with PLGA 65:35 blends after a slightly longer period, and degradation was not seen with PLGA 75:25 blends up through over four weeks. Bioerosion therefore is predicted to be proportional to the amount of glycolic acid in the PLGA co-polymer. The steroidal anti-inflammatory drug triamcinolone would tend to decrease faster degradation due to bioerosion by inhibiting the inflammatory response, therefore ensuring relatively prolonged release of the active water-soluble drug molecule (e.g. nalmefene). Further, the present invention teaches that release rate can be maintained at higher levels by minimizing thickness of the mold or putty-like sample (“putty”), or increasing its surface area relative to its volume. For example, the tablets just described were 3 mm thick. Cylinders formed by extrusion of the putty through a hollow needle would yield such a result. Even the more viscous putty with one-half the ratio of triacetin is easily formed into various physical shapes by the process of extrusion. Triacetin is a preferred plasticizer because it has been used parenterally in animals without adverse effects, and has been approved for human use as a pharmaceutical plasticizer for non-parenteral uses. For example, Tarr, Sambandam and Yalkowsky writing in “Pharm Res 1987 April;4(2):162-5” in an article titled “A new parenteral emulsion for the administration of Taxol” teach that emulsions for drug delivery made of 50% triacetin can be safely injected intravenously into mice. Bailey, Heath and Miles in “Am J Clin Nutr 1989 February;49(2):385-8” teach that the short chain triglyceride triacetin may be used as a parenterally administered nutrient with no demonstrable adverse effects on calcium, magnesium or phosphorus metabolism in dogs (see abstract “Calcium, magnesium, and phosphorus metabolism in dogs given intravenous triacetin”). Bailey, Haymond and Miles in “JPEN J Parenter Enteral Nutr 1991 January-February;15(1):32-6 write in “Triacetin: a potential parenteral nutrient” that triacetin is safely infused intravenously in dogs. Karlstad, Killeffer, Bailey and DeMichele in “Am J Clin Nuir 1992 May;55(5):1005-11” teach that triacetin as a component of total parenteral nutrition, also known generally as “TPN,” is safe and effective in rats. Likewise, Bleiberg, Beers, Persson and Miles writing in “Am J Clin Nutr 1993 December;58(6):908-11” teach that triacetin may be used as a form of intravenous nutrition in dogs. Subsequent studies have shown similar utility of triacetin. To this author's knowledge, the present invention is the first in which triacetin is used as a plasticizer for in vitro or ex vivo formation of a pharmaceutical delivery system intended for subcutaneous administration. Thus, this too represents a novel, unique and unobvious component of the present invention.

The above examples are for illustrative purposes only, and are not intended to limit the scope of the invention in any way. In light of the present invention that was brought about by experimentation, and to some degree by fortuitous circumstance, other embodiments of the invention may be apparent to those skilled in the art. Such embodiments may come after additional experimentation, but nonetheless experimentation that is usual and typical of the kind carried out in formulating and testing pharmaceutical preparations. Such examples have been mentioned herein, such as altering the PLGA used, altering the ratio of a particular PLGA to a particular plasticizer, etc. Also the concentration and ratio of active drugs, such as nalmefene, buprenorphine, nalbuphine and other water soluble drugs of similar molecular weight, ionic charge and so forth, can be manipulated to yield a drug release that would be optimal for that particular active drug based on characteristics of the particular drug such as it's molecular structure, volume of distribution, lipid solubility, elimination half-life, biological half-life, pK, pH of the surrounding medium, physical stability of the drug in a medium, etc. The release rate of active water-soluble drug has also been found able to be altered by limiting or eliminating PEG from the final product, as PEG because of its physical characteristics promotes swelling. The present invention also teaches that blending PLGA 75:25 copolymer with PLGA 50:50 copolymer may limit swelling compared to 75:25 PLGA alone, while limiting degradation compared to PLGA 50:50 alone. What is novel and new is the mechanism of release of drug, along with its resultant characteristics relating to rate of drug release, rate of change of drug release, lack of drug burst, and the way these characteristics may relate to the disease entities for which the active drugs are intended to treat. It is of further great utility that administration by simple subcutaneous needle injection is easy, practical and does not involve more invasive surgical procedures such as formed pellet implantation. Further embodiments of the invention will become apparent in light of the teachings that follow.

Gekker, Lokensgard and Peterson in Drug and Alcohol Dependence 64 (2001), pages 257-263 describe in “Naltrexone potentiates ant-HIV 1 activity of antiretroviral drugs in CD4+ lymphocyte cultures” how naltrexone acts to increase the anti-HIV effects of drugs used to treat HIV infection and AIDS. Li, Wang, Tian et al. in Journal of Infectious Disease, January. 1;185(1):118-22 teach in “Methadone enhances human immunodeficiency virus infection of human immune cells” that the opioid agonist methadone increase HIV virus activation and replication. These authors reiterate their findings in the Journal of Infectious Disease January 1, 2002;185(1):118-22 in another article with the same title. Mahayni and Minor wrote a letter in the American Journal of Hospital Pharmacy November;48(11):2480-1 stating that “research data suggests that the narcotic antagonists naltrexone and naloxone may possess anti-HIV activity,” but they make no mention of nalmefene. Dr. Bernard Bihari wrote a letter in AIDS Patient Care 1995 February;9(1).3 observing that trials of low-dose naltrexone with AIDS patients showed that the naltrexone was associated with significant and advantageous differences in the incidence of opportunistic infections, and that AIDS patients administered naltrexone maintained “good” CD4 lymphocytes. He intimates that this may be due to the immunological role of endorphins as “key hormones” in regulating the immune system. Schluger, Ho, Borg et al. in Alcohol Clinical Experimental Research 1998 October;22(7):1430-6 in their article “Nalmefene causes greater hypothalamic-pituitary-adrenal activation than naloxone in normal volunteers: implications for treatment of alcoholism” demonstrate that “kappa- and delta-opioids may play important roles in the regulation of the hypothalamic-pituitary-adrenal axis.” (They do not discuss any possible role related to HIV). Suzuki, Chuang, Chuang et al. in Advances in Experimental Medicine and Biology 2001,493:81-7 in a chapter titled “Morphine upregulates kappa-opioid receptors of human lymphocytes” teach that “chronic morphine use also induces immunomodulatory and immunosuppressive effects, as especially evident in HIV-infected patients,” and that this phenomenon involves kappa-opioid receptors. This, taken together with the advantageous opioid receptor subtype binding profile of nalmefene as described herein and which was noted prior to October 1998 in U.S. patent application Ser. No. 08/643,775 filed May 6, 1996 supports the present invention that teaches nalmefene as a preferential treatment and prophylactic medication for HIV viral infection and AIDS. The present invention also teaches the manufacture and use of a pharmaceutical preparation for preventing HIV infection as a prophylactic measure and for treating HIV infection in HIV-infected individuals and those with AIDS.

The present invention also teaches the superiority of nalmefene for treatment of alcoholism compared to other opioid antagonists due to a decreased tendency for cardiac dysrhythmias in alcoholic patients at increased risk for such dysrhythmias that was not previously appreciated by those skilled in the art. Nalmefene has been used to treat alcoholism, most notable by Dr. Barbara Mason in Florida. However, it has really been used in the prior art as an analogous compound to naltrexone, which the invention at hand clearly demonstrates it is not. In addition to the distinguishing characteristics of nalmefene demonstrated previously herein, as it relates to a drug addiction when the drug abused is ethanol (“alcohol”) the following advantages of nalmefene are encompassed in the present invention. Smetnev, Gorgaaslidze, Zinkin et al. in “Terk Arkh 1988;60(1):49-51” [orginial article in Russian], point out that more than 29% of alcoholic patients have cardiac abnormalities manifesting as “arrhythmical paroxysms.” Faintuch in “Rev Hosp Clin Fac Med Sao Paulo 1995 January-February;50(1):76-9” [original article in Portuguese] states that “both acute and chronic alcohol consumption precipitate arrhythmias.” Fabrizio and Regan in “Cardiovasc Drugs Ther 1994 February;8(1):89-94 report in their article “Alcoholic cardiomyopathy” that “atrial arrhythmias have been shown to occur during the early ethanol withdrawal phase in patients without other clinical evidence of heart disease.” It is common knowledge in medical practice that “holiday heart syndrome” consists of cardiac dysrhythmia due to the high ingestion of ethanol around the time of holiday celebrations, which quite unfortunately is sometimes fatal. Actions of alcohol inducing dysrhythmias on a cellular level have been described in animal models. For instance, Nakamura, Houchi, Ohe an Namba in Alcohol Clinical Experimental Research 1999 April;23(4 Suppl):81S-84S teach in their article “Increase in beating rate of cultured chick cardiac myocytes by ethanol and inhibition of the increase by antiarrhythmic drugs” that “drinking alcohol sometimes causes cardiac arrhythmia.” Going back to 1976, Ettinger et al. teach in the American Heart Journal 1976 January;91(1):66-78 in their article “Cardiac conduction abnormalities produced by chronic alcoholism” that “cardiac conduction abnormalities and rhythm disturbances are common clinical findings” in alcoholic patients with manifestations of long-term alcohol consumption. Those skilled in the art of treating alcoholic human patients clinically have failed to appreciate what is demonstrated by Caldwell, Nagarajan, Chryssanthis and Tuttle in Pharmacology 1990;41(3):161-6 in their articled titled “Actions of the opioid antagonist, nalmefene, and congeners on reperfusion cardiac arrhythmias and regional left coronary blood flow.” Caldwell et al. reach that nalmefene “reduced the incidence of reperfusion arrhythmias significantly when compared to the saline control,” and that “neither N-methyl-nalmefene . . . nor (+)nalmefene . . . provided any protection against reperfusion arrhythmias.” They concluded that nalmefene prevents the occurrence of such arrythmias. These studies, taken within the context of treating human alcoholics with opioid antagonists, support the present invention that nalmefene, as distinguished from other opioid antagonists used in treatment of drug addiction (e.g., naltrexone), is a preferred drug in the treatment of alcoholic patients. This, not being obvious to others skilled in the art, has not been attributed to nalmefene in the context of clinical trials using nalmefene to treat alcoholism. The present invention teaches that nalmefene, being non-analogous to naltrexone, is a preferred drug for the treatment of alcoholism. Schluger et al (ibid) do not make the case for nalmefene as being a preferred drug for treatment in alcoholism. In fact, they state merely “the effects of nalmefene and also naltrexone on modulating the tonic inhibition exerted by endogenous opioids acting at kappa- and delta-, as well as mu-, opioid receptors on the hypothalamic-pituitary-adrenal (“HPA”) axis may be related to their [emphasis added] established efficacy as treatment agents for alcoholism (see page 1434, Ibid). Further, with regard to nalmefene being a non-analogous preferred agent to naltrexone for alcoholism, Schluger et al. only conclude “nalmefene, as well as other kappa and perhaps delta-opioid antagonists and agonists, may therefore be useful tools to further elucidate some of the basic physiology and pathophysiology of the HPA axis, the endogenous opioid system, the biology of addictions, and the intersections between them” (see page 1435, Ibid). Such vague and convoluted language does not teach nalmefene as a preferred opioid antagonist to treat alcoholism, therefore the present invention is not obvious to one of ordinary skill in the art because of Schluger et al.

The present invention also teaches that 6-beta-naltrexol is a preferred opioid antagonist for the treatment of addictions, most notably opioid addiction, as is nalbuphine. Wang, Raehal, Blisky and Sadee in their article “Inverse agonists and neutral antagonists at mu opioid receptor (MOR): possible role of basal receptor signaling in narcotic dependence” in the Journal of Neurochemistry 2001 June; 77(6):1590-600, teach that the neutral opioid antagonist 6-beta-naltrexol possesses certain important advantages over other opioid antagonists in that 6-beta-naltrexol does not inhibit intrinsic mu-opioid receptor “agonist-like activity” to the degree that the opioid antagonists naltrexone, naloxone and nalmefene do. This is important in a previously opioid-dependent patient recently detoxified and withdrawn from opioids to which a sustained release opioid antagonist is administered. Clinical evidence indicates that naltrexone pellets implanted into humans as part of a detoxification procedure is associated with substantial withdrawal related signs and symptoms in the post-detoxification period. Accordingly, 6-beta-naltrexol would not cause the same degree of opioid withdrawal related symptoms in the post detoxification period if it were administered in lieu of naltrexone.

The present author has previously quite unexpectedly discovered that nalbuphine, also known by the trade name Nubain® (Endo Pharmaceuticals, Chadds Ford, Pa.), reverses detrimental effects of fentanyl such as respiratory depression without totally inhibiting muopioid agonist-like activity. The present author has also quite surprising discovered that nalbuphine, although precipitating opioid withdrawal in actively opioid-dependent patients due to antagonist effects, also relieves opioid withdrawal symptoms in patients in the immediate post-detoxification period. Thus, by incorporating nalbuphine hydrochloride in a putty-like semi-sol described herein for administration and sustained delivery of the water-soluble nalbuphine molecule, a method of blocking opioid receptors from “pure” opioid agonist analgesics, such as morphine, heroin or fentanyl is manifested, while simultaneously allowing for some mu-opioid agonist-like activity. Thus, the present invention recognizes nalbuphine as a partial mu-opioid agonist, which may be interpreted that it is also a partial mu-opioid antagonist, and at the very least allows for intrinsic mu-opioid receptor activity.

The present invention also provides a structural composition comprising a therapeutic dose of opioid agonist analgesic in combination with an amount of 6-beta-naltrexol or nalbuphine effective to allow for the positive effects of the opioid agonist analgesic, while at the same time exerting relatively less antagonistic effects at mu-opioid receptors compared to other opioid antagonists, when the opioid agonist analgesic is administered in recommended therapeutic doses, such that the agonist actions of the opioid agonist analgesic will far outweigh any antagonism by 6-beta naltrexol or nalbuphine at said mu-opioid receptors. If excessive amounts of the structural composition comprising 6-beta naltrexol or nalbuphine and opioid agonist analgesic are administered, enough 6-beta naltrexol or nalbuphine shall be administered as to begin to antagonize or block mu-opioid receptors from the exogenously administered opioid agonist analgesic, while simultaneously allowing for some intrinsic muopioid agonist-like activity. Thus, excessive opioid agonist analgesic is blocked, preventing overdose or excessive euphoric effects, while the likelihood of triggering a withdrawal response is greatly diminished.

### EXAMPLE 24

A recommended therapeutic dose of morphine, e.g. 0.15 mg/kg morphine, preferably in the form of morphine sulfate, is co-administered parenterally with 0.00025 to 0.0015 milligrams per kilogram (mg/kg) 6-beta naltrexol, preferably in the form of 6-beta naltrexol hydrochloride, more preferably 0.0007 mg/kg 6-beta naltrexol. For a young adult 70 kg human, for example, 10.5 mg morphine sulfate is administered parenterally, along with 0.049 mg, or 49 micrograms (ug), 6-beta naltrexol hydrochloride parenterally. This small amount of 6-beta naltrexol, consistent with the present invention, produces minimal appreciable effect at mu-opioid receptors in relation to the 10.5 mg dose of morphine.

In a preferred embodiment of the present invention, morphine sulfate and 6-beta naltrexol hydrochloride are co-existent in a common medium compatible for parenteral administration in the ratio, of 0.15 mg active morphine to 0.0007 mg active 6-beta naltrexol. Ideally, if administered subcutaneously, the total amounts of the two co-administered active drugs would be contained within an injectable volume of approximately 1 to 2 milliliters (cc) or less for a 70 kg adult human.

### EXAMPLE 25

Among the most commonly written prescriptions in the United States, are the prescriptions for combination oral analgesics consisting of an opioid agonist analgesic and a non-opioid analgesic such as acetaminophen, aspirin, ibuprofen or other non-steroidal anti-inflammatory drug (“NSAID”). By way of example only, the combination of oxycodone and acetaminophen, commonly known by the brand name Percocet®, is very often prescribed for a wide variety of pain syndromes, including pain secondary to surgery or trauma, and malignancies. Similarly, the drug formulation commonly known by the brand name Percodan® is composed of oxycodone and aspirin, and the opioid agonist analgesic hydrocodone in its bitartrate form is combined with the non-opioid analgesic acetaminophen.

Orally administered combination drugs consisting of an opioid agonist analgesic and another drug(s) or medication(s) are among the most widely abused opioid agonists abused. If these combination drugs contain acetaminophen, as in the case with Percocet®, a large amount of Percocet® tablets may be orally ingested, so much so as to cause a toxic load of acetaminophen to be delivered. Acetaminophen is widely known to be toxic to the liver of humans when administered in excessive dosages, or when abused by self-administration either intentionally or unintentionally. Often, because of the tolerance built up to the opioid agonist analgesic component of the Percocet®, the patient will progressively ingest more and more Percocet® tablets over time in an attempt to satisfy the effect of the opioid agonist analgesic at mu opioid receptors. Because of the stealth adverse effects of acetaminophen toxicity, often a human may not be aware of harm caused to him or her by the large ingestion of acetaminophen in the combination drug formulation, until a medical exam reveals abnormal liver function, or until liver failure suddenly becomes apparent. Compounding this problem is the fact that orally administered drugs are delivered relatively directly to the liver, by a mechanism commonly called “first pass metabolism.” Thus, there is presently a great need to formulate an orally administrable combination drug therapy that would allow for the additive or synergistic effects of combining analgesics, such as opioids and acetaminophen, but that could prevent or strongly limit the likelihood of developing liver failure secondary to liver toxicity.

In the case of Percodan®, a large amount of Percodan® tablets may be orally ingested, so much so as to cause a toxic load of aspirin to be delivered. Aspirin and other NSAIDs are widely known to cause gastrointestinal bleeding of humans when administered in excessive dosages. Often, because of the tolerance built up to the opioid agonist analgesic component of the Percodan®, the patient will progressively ingest more and more Percodan® tablets over time in an attempt to satisfy the effect of the opioid agonist analgesic at mu opioid receptors. Because of the unobvious early signs of adverse effects of aspirin or NSAID toxicity, often a human may not be aware of harm caused to him or her by the large ingestion of aspirin or NSAID in the combination drug formulation, until a medical exam reveals abnormal gastric function, or until an acute gastric hemorrhage suddenly becomes apparent. Compounding this problem is that when aspirin or other NSAIDs are administered orally, or per os, they are delivered directly to the stomach through the esophagus where their breakdown begins to occur in direct contact with the gastric mucosa lining the stomach. This is the very site of the caused gastric bleeding. Thus, there is presently a great need to formulate a orally administrable combination drug therapy that would allow for the additive or synergistic effects of combining analgesics, such as opioids and NSAIDs (of which aspirin is an example), but that could prevent or strongly limit the likelihood of developing gastrointestinal bleeding.

Hydrocodone (as hydrocodone bitartrate, for example) and other opioid agonist analgesics are commonly mixed with other non-opioid analgesic drugs in formulating combination medications.

By way of example only, if a combination medication tablet that is formulated with 10 milligrams (mg) of hydrocodone and 0.2 mg nalbuphine (=200 micrograms) is ingested orally, only about 30 microgram (mcg) of nalbuphine will be delivered unmetabolized to the bloodstream. A formulation proportionate to 10 mg hydrocodone and 100 mcg nalbuphine (0.1 mg), perhaps mixed with acetaminophen 500 mg, would be expected to produce analgesia not significantly or very appreciably different from a formulation of 10 mg hydrocodone and 500 mg acetaminophen without nalbuphine. However, if a human were to ingest two such formulated tablets every four hours, as commonly occurs when human patients self-administer these medications in larger than doses prescribed or intended by a physician, then over a 12-plus hour period a human would ingest 8 tablets comprising 80 mg hydrocodone and 0.8 mg nalbuphine. Because the plasma half-life of both hydrocodone and acetaminophen is approximately 3 hours, and the plasma elimination half-life of nalbuphine is approximately 3.5 hours, nalbuphine will tend to accumulate over time relative to hydrocodone and acetaminophen such that as more time progressively transpires the nalbuphine serum concentration relative to hydrocodone serum concentration will increase as the tablets are ingested over that time. Further, by administering the medication every 4 hours, steady state concentration of nalbuphine will occur in approximately 24 hours. Eventually, this will cause an appreciably different effect of the opioid agonist analgesic. This effect could include prevention of mortal respiratory depression, or lack of satisfaction due to opioid ingestion. The exact nature of this interaction is easily altered by one skilled in the art by changing the relative amounts of nalbuphine to opioid agonist analgesic in the tablet, as well as by altering the pharmacokinetic profile of either drug by including a sustained release preparation of either the nalbuphine or the opioid agonist analgesic. This would be calculated during the normal course of experimentation routine for deriving such data. Such experimentation is routinely employed in formulating pharmaceutical preparations to required standards. The dosages are thus described generally in terms of pharmacological effect.

Though hydrocodone and oxycodone are mentioned by way of example here, the scope of the invention encompasses any orally administered opioid agonist analgesic. Such applicable opioid agonist analgesics include the following opioids and their derived salts and bases: morphine, propoxyphene, fentanyl, methadone, levomethadyl (LAAM) and codeine.

I claim by Letters Patent:

1.) A drug delivery system comprising a biodegradable copolymer, a plasticizer, and an effective amount of a water-soluble drug.

2.) The drug delivery system of claim 1 where the biodegradable copolymer is PLGA.

3.) The drug delivery system of claim 1 where the plasticizer is triacetin.

4.) The drug delivery system of claim 1 where the water-soluble drug is an opioid antagonist.

5.) The drug delivery system of claim 4 where the opioid antagonist is nalmefene.

6.) A drug delivery system comprising a biodegradable copolymer, a plasticizer, an effective amount of a water-soluble drug, and a steroidal anti-inflammatory drug.

7.) The drug delivery system of claim 6 where the steroidal anti-inflammatory agent is triamcinolone.

8.) A method of treating alcoholism comprising administration of nalmefene.

9.) A method of treating HIV infection comprising administration of an opioid antagonist.

10.) The method of claim 9 where the opioid antagonist is nalmefene.

11.) A pharmaceutical composition comprising an opioid agonist analgesic and 6-beta-naltrexol.

12.) A pharmaceutical composition comprising an opioid agonist analgesic and nalbuphine.

13.) The drug delivery system of claim 4 where the opioid antagonist is 6-beta-naltrexol.

14.) The drug delivery system of claim 4 where the opioid antagonist is nalbuphine.

15.) The drug delivery system of claim 1 where the water-soluble drug is buprenorphine.
